(19) United States
(12) Patent Application Publication
(10) Pub. No.: US 2022/0354983 A1
(43) Pub. Date: Nov. 10, 2022

(54) QUARTZ CRYSTAL UV LIGHT

(71) Applicant: Biozone Scientific Group Limited, London (GB)

(72) Inventors: Adam Anthony, Windermere, FL (US); McKay Howell, Kissimmee, FL (US); John Phillips, Orlando, FL (US); Ari Ahola, Windermere, FL (US)

(73) Assignee: Biozone Scientific Group Limited, London (GB)

(21) Appl. No.: 17/302,590

(22) Filed: May 7, 2021

(51) Int. Cl. A61L 9/20

(52) U.S. Cl. CPC A61L 9/205; A61L 2209/12; A61L 2209/16

(57) ABSTRACT

A germicide system for use in an air duct including a lamp, a lamp housing, and a control unit. The lamp housing is adapted to be positioned in an interior of the air duct and includes a lamp shield and quartz wool carried by the lamp shield. The lamp is carried by the lamp housing. The control unit is adapted to be positioned entirely in an exterior of the air duct and adapted to provide power to the lamp.

### FIELD OF THE INVENTION

The present invention relates to systems and methods for disinfecting air. More specifically, the present invention is directed to a system for disinfecting air within an air duct.

### BACKGROUND OF THE INVENTION

Some of the earliest published references to titania (titanium dioxide or TiO₂) photocatalysts are by Formenti; M., et al., “Heterogeneous Photocatalysis for Oxidation of Paraffins”, Chemical Technology 1, 680-686, 1971 and U.S. Pat. No. 3,781,194 issued Dec. 25, 1973. Since the 1972 discovery of the photocatalytic splitting of water on titanium dioxide electrodes, by Fujishima and Honda, the science and technology related to heterogeneous photocatalysis in both water and air has been extensively studied and is the subject of numerous patents and scientific publications. Both the physics and chemistry of heterogeneous photocatalysis remain areas of active investigation. Much of the early work of relevance to this patent is summarized, by Kittrell in U.S. Pat. Nos. 6,179,972 and 6,221,259, as well as, Peill, et al., in U.S. Pat Nos. 5,875,384 and 6,051,194. Despite investigation of many alternatives, the anatase crystal morphology of titanium dioxide remains the photocatalytically active semi-conductor of economic choice, although many claims of additive enhancements have been and continue to be made.

Considerable historical effort has been expended to maximize photocatalytic activity of anatase TiO₂ by minimizing particle size (to maximize effective surface area) while maintaining strong adhesion to substrate surfaces. It is well known and documented that photocatalytic activity is directly related to (a) the intensity and wavelength of irradiation at the illuminated photocatalyst surface area, (b) the magnitude of the illuminated photocatalyst surface area, (c) the rate of flow of contaminants across the illuminated photocatalyst surface area (irradiated surface contact), and (d) in air, the “absolute” humidity of the ambient air. The “quantum or photocatalytic efficiency” relates to the fraction of light-source photons that are effective in causing the photocatalyzed reactions.

Considerable effort is currently being expended, in the field of photocatalysis, to enhance the photocatalytic efficiency of anatase titanium dioxide with various catalytic additives (as described in many patents, e.g., U.S. Pat. Nos. 8,409,928, 6,179,972, and 6,221,269).

The objective of the present invention is to provide a large illuminated photocatalyst surface area while ensuring intimate air-surface contact, at all airflow rates, so as to maximize the photocatalytic efficiency of the light source. Where the photocatalyst substrate fibers are high-purity quartz, wool, mat, or felt, the fibers act as elementary waveguides having a UV-conductive core. Refraction and reflection (both internal and external) of randomly oriented fibers ensure efficient distribution of light photons throughout the fiber mass.

Another object of the present invention is to provide a disinfecting apparatus that is configured to be deployed in air ducts within an HVAC system to maximize disinfecting properties of the photocatalyst surface while minimizing maintenance of the system.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a germicide system for use in an air duct. The germicide system includes a lamp housing, a lamp, and a control unit. The lamp housing may be adapted to be positioned in an interior of the air duct and include a lamp shield and quartz wool carried by the lamp shield. The lamp may be carried by the lamp housing. The control unit may be adapted to be positioned entirely in an exterior of the air duct and adapted to provide power to the lamp.

The lamp may emit a light having a wavelength between 10 nanometers and 380 nanometers.

The quartz wool may be positioned to provide an air gap between an entirety of the quartz wool and the lamp.

The lamp shield may have a plurality of apertures located through an entirety of a thickness of the lamp shield.

The lamp shield may have an interior surface proximate the lamp. The interior surface may include a first planar surface located in a first plane, a second planar surface located in a second plane, and a third planar surface located in a third plane. The first plane and the second plane may intersect the third plane.

The quartz wool may include a first piece of quartz wool secured to the first planar surface, a second piece of quartz wool secured to the second planar surface, and a third piece of quartz wool secured to the third planar surface.

The lamp shield may further include a first plurality of apertures located through an entirety of a thickness of the first planar surface, a second plurality of apertures located through an entirety of a thickness of the second planar surface, and a third plurality of apertures located through an entirety of a thickness of the third planar surface.

The lamp housing may further include a photocatalyst embedded in the quartz wool.

The lamp housing may further include a plurality of carbon pellets embedded in the quartz wool. The photocatalyst may be titanium dioxide. The photocatalyst may not be directly affixed to the lamp.

The control unit may include a ballast adapted to regulate the power provided to the lamp and a control housing adapted to carry the ballast.

The lamp housing may further include a mesh shield secured to a first side and a second side of the lamp shield and forming an envelope defined by the mesh shield and the lamp shield, the lamp may be carried within the envelope.

The control unit may include a power supply.

The control unit may include a connector adapted to receive power from an external power supply.

The control unit may include an LED adapted to provide a status indicator.

The control housing may include a plurality of mounting apertures. Each of the plurality of mounting apertures may be adapted to receive a fastener to secure the control housing to the lamp housing.

### BRIEF DESCRIPTION OF THE DRAWINGS

Some embodiments of the present invention are illustrated as an example and are not limited by the figures of the accompanying drawings, in which like references may indicate similar elements.

FIG. 1 is a front perspective view of a germicide system according to an embodiment of the present invention.

FIG. 2 is a rear perspective view of the germicide system of FIG. 1.

FIG. 3 is an exploded view of the germicide system of FIG. 1.

FIG. 4 is a perspective view of the lamp housing of the germicide system of FIG. 1

FIG. 5 is a cross-section of the lamp housing taken through line 5-5 of FIG. 4.

FIG. 6 is a perspective view of the lamp shield of the lamp housing of FIG. 4.

FIG. 7 is a bottom plan view of the lamp shield of FIG. 6.

FIG. 8 is a detail view of section 8 of FIG. 7.

FIG. 9 is a side elevation view of the lamp shield of FIG. 6.

FIG. 10 is a perspective view of the quartz wool of the lamp housing of FIG. 4.

FIG. 11 is a side elevation view of the quartz wool of FIG. 10.

FIG. 12 is a top plan view of the quartz wool of FIG. 10.

FIG. 13 is a perspective view of the mesh shield of the lamp housing of FIG. 4.

FIG. 14 is a perspective view of a base of the control housing of the germicide system of FIG. 1.

FIG. 15 is a top plan view of the base of FIG. 14

FIG. 16 is a front elevation view of the base of FIG. 14.

FIG. 17 is a first side elevation view of the base of FIG. 14.

FIG. 18 is a second side elevation view of the base of FIG. 14.

FIG. 19 is a perspective view of a cover of the control housing of the germicide system of FIG. 1.

FIG. 20 is a side elevation view of the cover of FIG. 19.

FIG. 21 is a top plan view of the cover of FIG. 19.

FIG. 22 is a perspective view of a duct mounting adapter plate of the control housing of the germicide system of FIG. 1.

FIG. 23 is a perspective view of a duct gasket of the control housing of the germicide system of FIG. 1.

FIG. 24 Is a perspective view of an enclosure gasket of the control housing of the germicide system of FIG. 1.

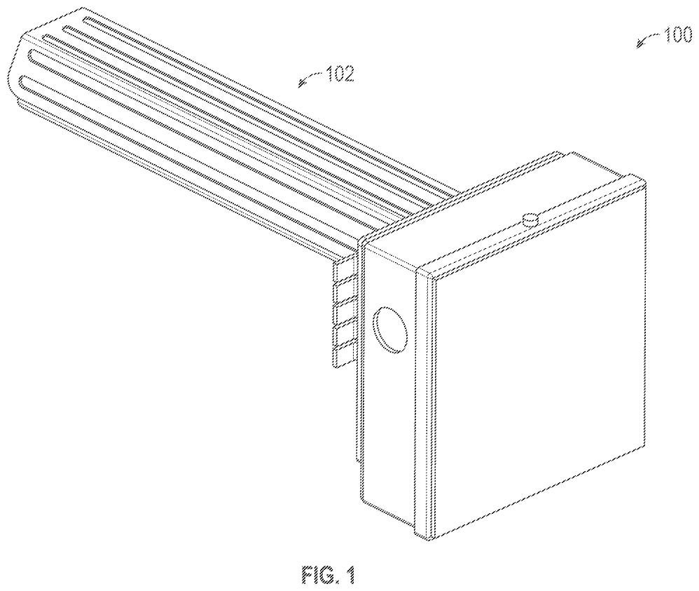

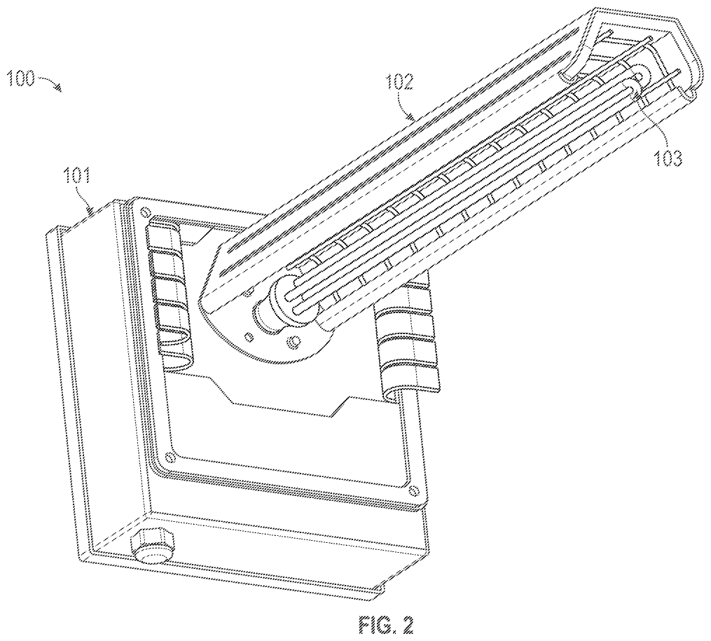

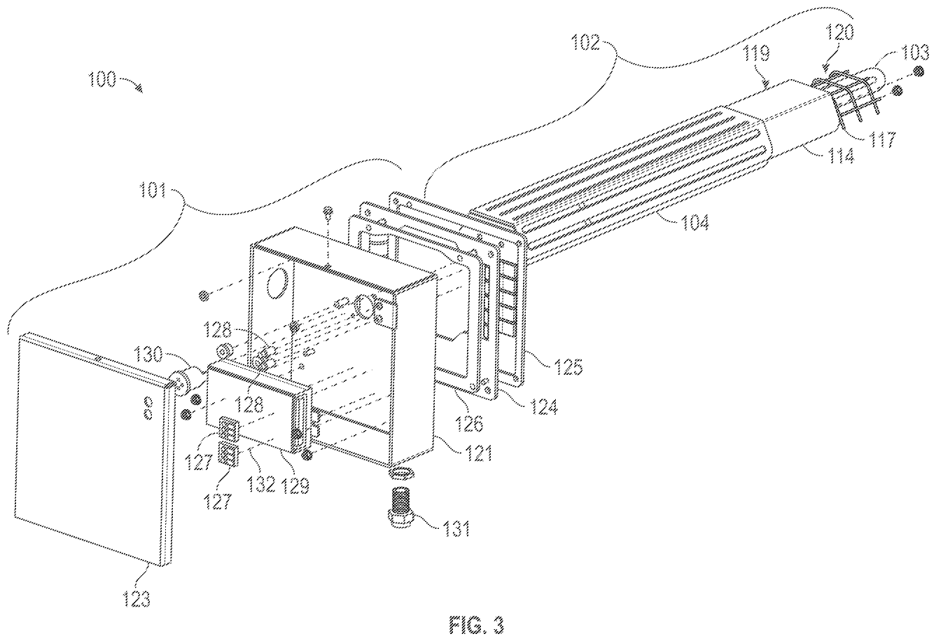

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a germicide system 100. The germicide system 100 may be particularly suited for use in an air duct of an HVAC system. The germicide system 100 may include a lamp housing 102, a lamp 103, and a control unit 101.

The lamp 103 may be carried by the lamp housing 102 and positioned within an interior of an air duct. The lamp 103 may be adapted to emit a wavelength in the UV spectrum. Specifically, the lamp may emit a light having a wavelength between 10 nanometers and 380 nanometers.

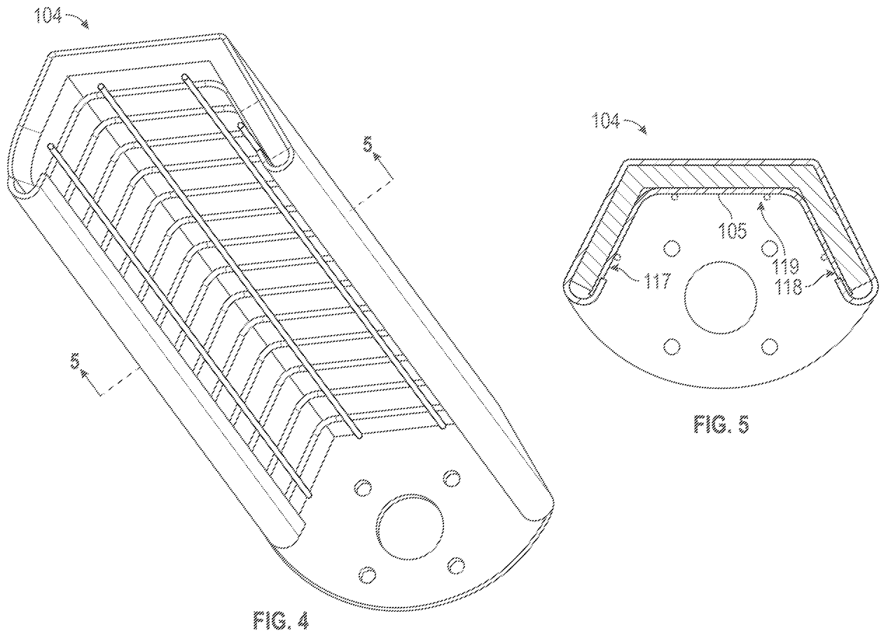

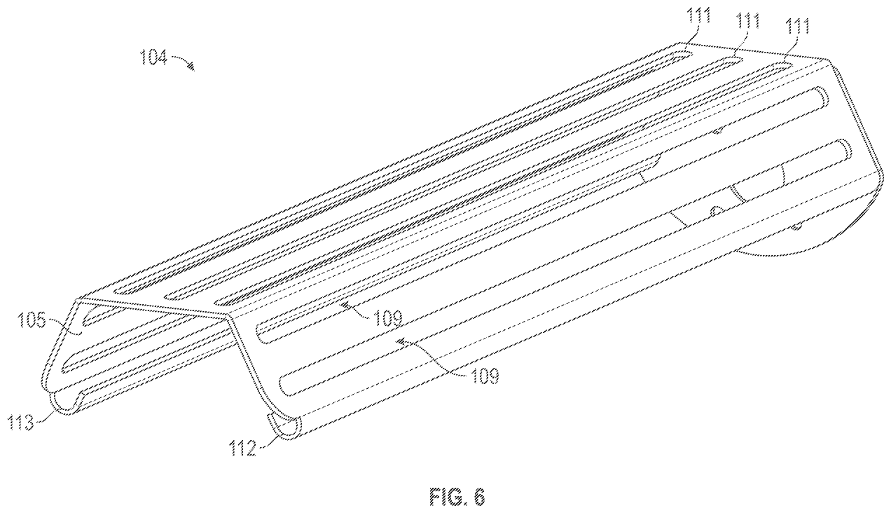

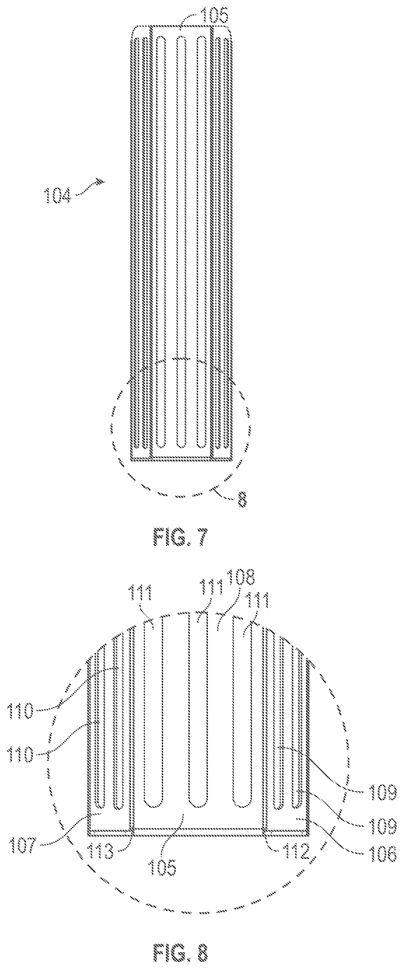

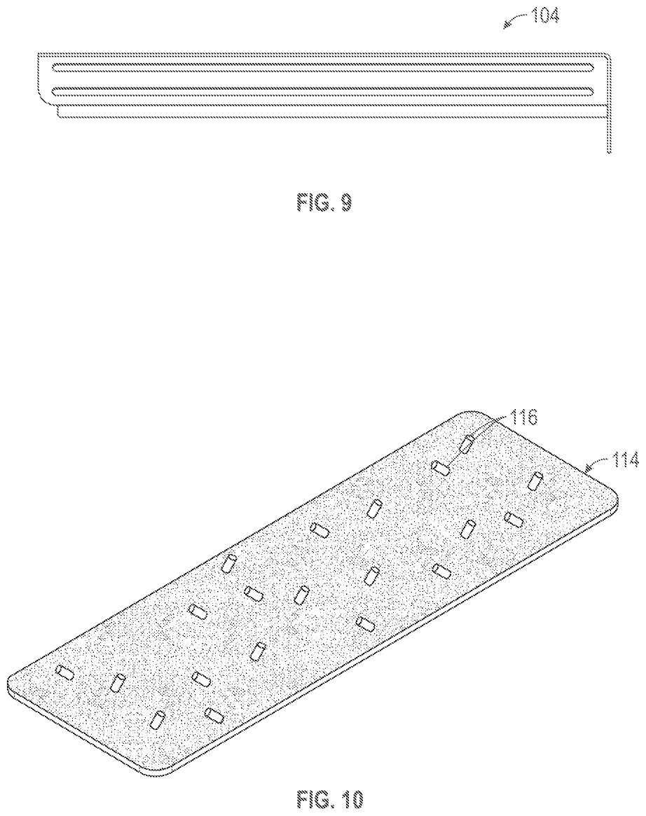

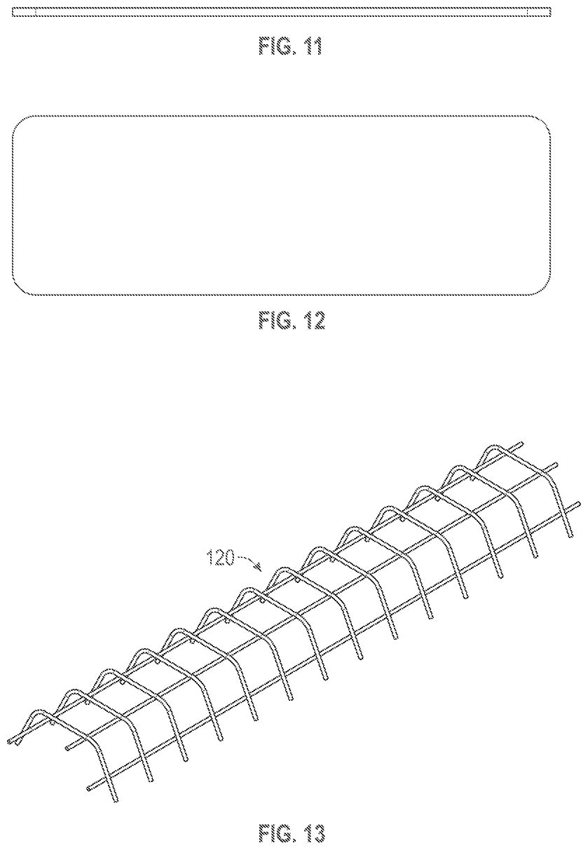

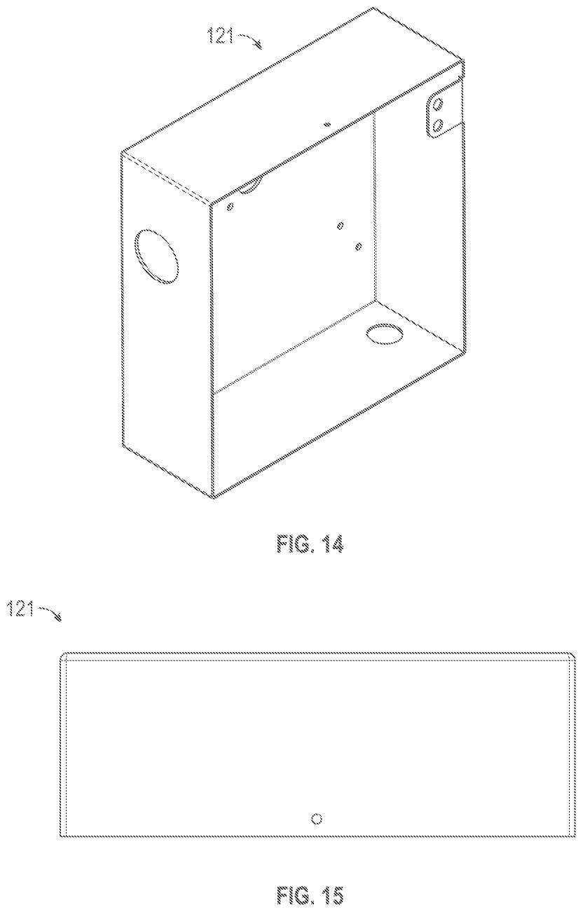

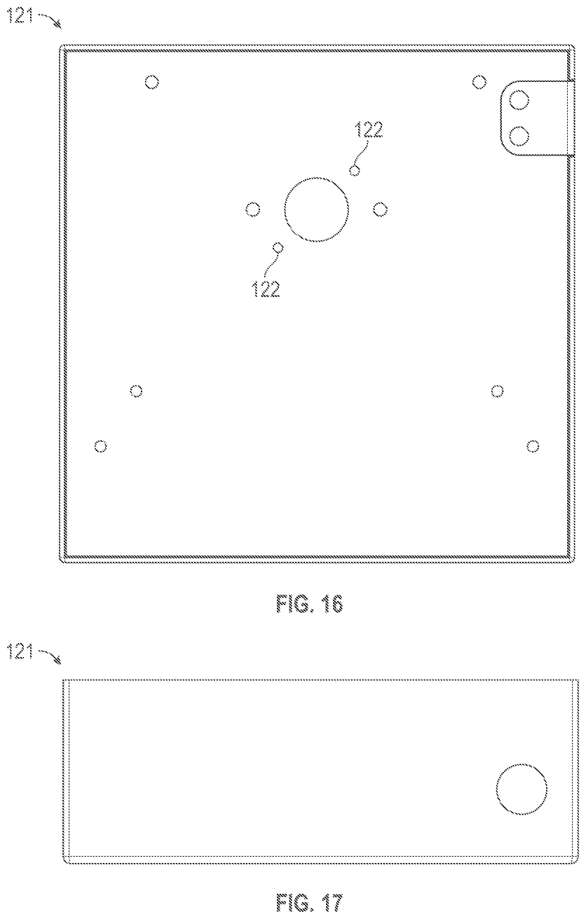

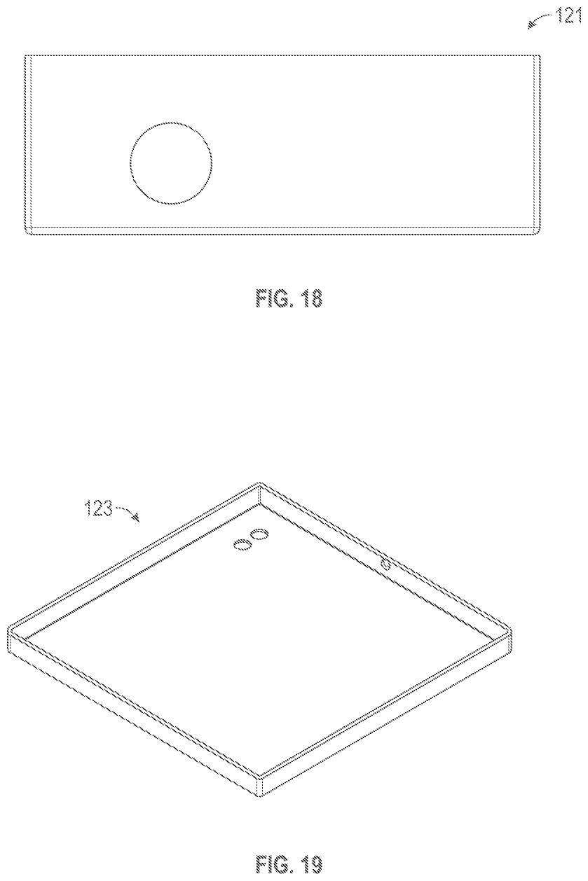

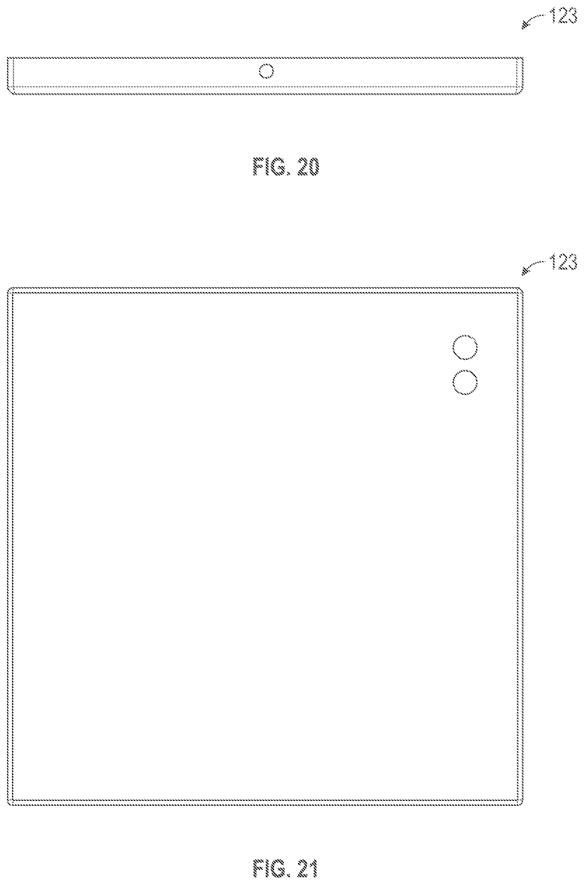

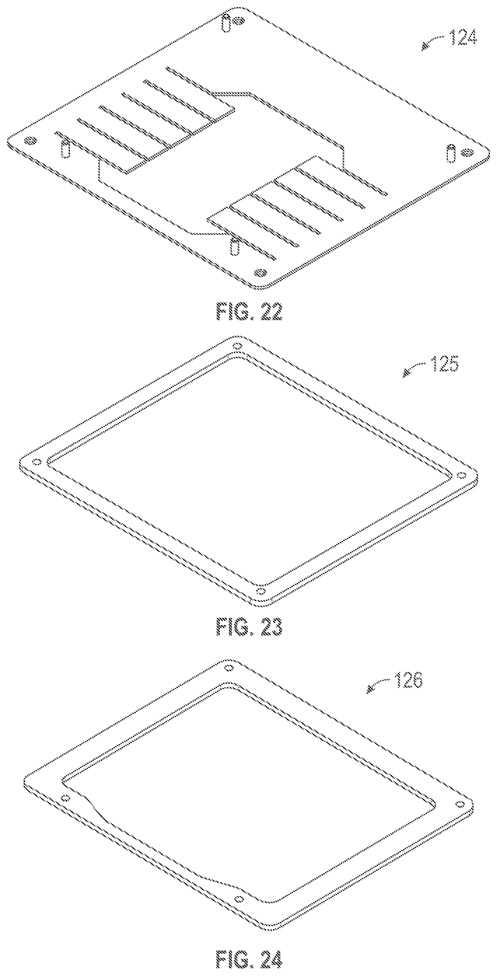

FIGS. 4-13 illustrate elements of the lamp housing 102. The lamp housing 102 may be adapted to be positioned in the interior of an air duct. The lamp housing 102 may include a lamp shield 104 and quart wool 114, which may be carried by and affixed directly to the lamp shield 104. The lamp shield 104 may have an inner surface 105 proximate the lamp 103. The quartz wool 114 may be affixed to the inner surface 105 of the lamp housing 102. The quartz wool 14 and lamp housing 102 may be positioned to provide an air gap between an entirety of the quartz wool 114 and the lamp 103. The air gap may allow air, and more specifically, particulates in the air, flowing through the duct to be exposed to the UV radiation and germicidal properties of the system. The air gap may beneficially increase the germicidal properties of the system 100.

The lamp shield 104 may secure to the control housing and extend away from the housing along a length of the lamp 103. The lamp shield 104 may be formed from three planar members. A central planar member 108 may secure to a second planar member 106 along a first side and may secure to a third planar member 107 along a second side. The angle between the interior surface of the central planar member 108 and both the second and third planar members 106, 107 may be less than 180 degrees and greater than 90 degrees. The angle between the central planar member 108 and the second planar member 106 may be the same as the angle between the central planar member 108 and the third planar member 107. Each planar member 106, 107, 108 may be located in a separate, non-parallel plane. The planes of the first and second planar members 106, 107 may intersect the plane of the central planar member 108 and may intersect with each other.

A first piece of quartz wool 117 may be secured to an interior surface of the first planar member 106. A second piece of quartz wool 118 may be secured to an interior surface of the second planar member 107. A third piece of quartz wool 119 may be secured to an interior surface of the third planar member 108. One or more pieces of quartz wool 114 may include photocatalyst 115 embedded within the quartz wool. The photocatalyst 115 may enhance the germicidal benefits of the system. In one embodiment, the photocatalyst 115 may be titanium dioxide. The photocatalyst may be carried by the lamp housing 102 and specifically by the quartz wool 114 and may not be in physical contact with the lamp 103.

In one embodiment, the quartz wool 114 may include a plurality of carbon pellets 116 embedded in the quartz wool 114. The carbon pellets 116 may remove odors from the air flowing through the duct.

A plurality of apertures may be located in the lamp shield 104. The plurality of apertures may beneficially allow air, and particulates carried by the air, to flow through the lamp shield 104 and not obstruct the flow of air through the duct. The plurality of apertures may be formed through an entirety of a thickness of the lamp shield 104. A first plurality of apertures 109 may be located through an entirety of a thickness of the first planar member 106. A second plurality of apertures 110 may be located through an entirety of a thickness of the second planar member 107. A third plurality of apertures 111 may be located through an entirety of a thickness of the third planar member 108.

The lamp housing 102 may also include a mesh shield 120. The mesh shield 120 may have a first side secured to a first side 112 of the lamp shield 104 and a second side secured to a second side 113 of the lamp shield 104. The mesh shield 120 and the lamp shield 104 may form an envelope with the lamp 103 positioned and carried within the envelope. The mesh shield 120 may be formed from rigid wires with a first set of wires extending from a first side to a second side of the lamp shield 104 and a second set of wires extending orthogonal to the first set along a length of the lamp shield 104. The first and second set of wires may be positioned and spaced to form a mesh with air gaps between parallel wires.

The control unit 101 may be adapted to be positioned entirely in an exterior of the air duct and may provide power to the lamp 103. The control unit 101 may include a ballast 132, a control housing, a power supply 129, a connector 127, and one or more LEDs 128.

The ballast may be adapted to receive power, regulate that power, and provide the power to the lamp 103. The control housing may be adapted to carry the ballast 132, power supply 129, connector 127, lamp housing 102, and one or more LEDs 128.

The power supply 129 may be one or more batteries carried by the control housing. The power supply 129 may provide an electric power signal to the ballast 132. The power supply 129 may provide sufficient power to illuminate the lamp 103 continuously for 365 days.

In one embodiment, the system 100 may not include a power supply. In such an embodiment, the control unit 101 may include a connector 127 adapted to receive one or more wires providing power to the system 100 from an external source. In such an embodiment, power received through the connector 127 may be provided to the ballast 132. A system 100 may have either a connector 127 or a power supply 129. In some embodiments, a system 100 may have both a connector 127 and a power supply 129.

The one or more LEDs 128 may be adapted to provide a status indicator to a user of the system 100. In one embodiment, an LED 128 may be illuminated to indicate the system has power sufficient to illuminate the lamp 103. In another embodiment, the LED 128 may be illuminated to indicate the system has encountered an error, the error may be, by way of example and not as a limitation, that the lamp 103 is unable to be illuminated.

The control housing may include a base 121 and a cover 123. The base 121 and cover 123 may be cooperatively configured to form an envelope between the two components. The ballast 132, power supply 129, connector 127, and LED 128 may be carried within the envelope.

A plurality of mounting apertures 122 may be formed through an entirety of a thickness of a wall of the base 121. Each of the plurality of mounting apertures 122 may be adapted to receive a fastener to secure the base 121 to the lamp housing 102. The fastener may be a screw, bolt, rivet, or the like. In one embodiment, the plurality of mounting apertures 122 may be located on a rear surface of the base 121 of the control housing and disposed around a central housing adapted to receive a lamp connector 130, which is adapted to provide a physical and mechanical connection to the lamp 103.

The system 100 may include a duct mounting adapter plate 124, a duct gasket 125, an enclosure gasket 126, and strain relief 131. The duct gasket 125 may be positioned adjacent a duct when the system 100 is installed and sandwiched between the duct and the duct mounting adapter plate 124. The duct gasket 125 may form a seal between the duct mounting adapter plate 124 and the duct. The enclosure gasket 126 may be sandwiched between the duct mounting adapter plate 124 and the base 121 of the control housing. The enclosure gasket 126 may form a seal between the base 121 of the control housing and the duct mounting adapter plate 124. Strain relief 131 may be carried by the base 121 and carry one or more electrical power wires when power is provided to the system 100 from an external source.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. A germicide system for use in an air duct comprising:
a lamp housing adapted to be positioned in an interior of the air duct and comprising:
a lamp shield, and
quartz wool carried by the lamp shield;
a lamp carried by the lamp housing; and
a control unit adapted to be positioned entirely in an exterior of the air duct and adapted to provide power to the lamp.

2. The system of claim 1 wherein the lamp emits a light having a wavelength between 10 nanometers and 380 nanometers.

3. The system of claim 1 wherein the quartz wool is positioned to provide an air gap between an entirety of the quartz wool and the lamp.

4. The system of claim 1 wherein the lamp shield has a plurality of apertures located through an entirety of a thickness of the lamp shield.

5. The system of claim 1 wherein the lamp shield has an interior surface proximate the lamp and the interior surface comprises:
a first planar surface located in a first plane;
a second planar surface located in a second plane; and
a third planar surface located in a third plane; and
wherein the first plane and the second plane intersect the third plane.

6. The system of claim 4 wherein the quartz wool further comprises:
a first piece of quartz wool secured to the first planar surface;
a second piece of quartz wool secured to the second planar surface; and
a third piece of quartz wool secured to the third planar surface.

7. The system of claim 5 wherein the lamp shield further comprises:
a first plurality of apertures located through an entirety of a thickness of the first planar surface;
a second plurality of apertures located through an entirety of a thickness of the second planar surface; and
a third plurality of apertures located through an entirety of a thickness of the third planar surface.

8. The system of claim 1 wherein the lamp housing further comprises a photocatalyst embedded in the quartz wool.

9. The system of claim 1 wherein the lamp housing further comprises a plurality of carbon pellets embedded in the quartz wool.

10. The system of claim 5 wherein the photocatalyst is titanium dioxide.

11. The system of claim 6 wherein the photocatalyst is not directly affixed to the lamp.

12. The system of claim 1 wherein the control unit comprises:
a ballast adapted to regulate the power provided to the lamp; and
a control housing adapted to carry the ballast.

13. The system of claim 1 wherein the lamp housing further comprises a mesh shield secured to a first side and a second side of the lamp shield and forming an envelope defined by the mesh shield and the lamp shield, wherein the lamp is carried within the envelope.

14. The system of claim 1 wherein the control unit comprises a power supply.

15. The system of claim 1 wherein the control unit comprises a connector adapted to receive power from an external power supply.

16. The system of claim 1 wherein the control unit comprises an LED adapted to provide a status indicator.

17. The system of claim 12 wherein the control housing comprises a plurality of mounting apertures, wherein each of the plurality of mounting apertures is adapted to receive a fastener to secure the control housing to the lamp housing.

18. A germicide system for use in an air duct comprising:
a lamp housing adapted to be positioned in an interior of the air duct and comprising:
a lamp shield having a thickness and a plurality of apertures located through an entirety of the thickness,
quartz wool carried by the lamp shield and positioned to provide an air gap between an entirety of the quartz wool and a lamp, and
a mesh shield secured to a first side and a second side of the lamp shield and forming an envelope defined by the mesh shield and the lamp shield, wherein the lamp is carried within the envelope;
the lamp carried by the lamp housing; and
a control unit adapted to be positioned entirely in an exterior of the air duct and adapted to provide power to the lamp, comprising.
a ballast adapted to regulate the power provided to the lamp, and
a control housing adapted to carry the ballast.

19. The system of claim 18 wherein the lamp emits a light having a wavelength between 10 nanometers and 380 nanometers.

20. A germicide system for use in an air duct comprising:
a lamp housing adapted to be positioned in an interior of the air duct and comprising:
a lamp shield having an interior surface proximate the lamp, a first planar surface in a first plane on the interior surface, a second planar surface in a second plane on the interior surface, and a third planar surface in a third plane on the interior surface, wherein the first plane and the second plane intersect the third plane,
a first plurality of apertures located through an entirety of a thickness of the first planar surface,
a second plurality of apertures located through an entirety of a thickness of the second planar surface,
a third plurality of apertures located through an entirety of a thickness of the third planar surface,
a first piece of quartz wool secured to the first planar surface and positioned to provide a first air gap between an entirety of the first piece of quartz wool and a lamp,
a second piece of quartz wool secured to the second planar surface and positioned to provide a second air gap between an entirety of the second piece of quartz wool and the lamp,
a third piece of quartz wool secured to the third planar surface and positioned to provide a third air gap between an entirety of the third piece of quartz wool and the lamp, and
a mesh shield secured to a first side and a second side of the lamp shield and forming an envelope defined by the mesh shield and the lamp shield, wherein the lamp is carried within the envelope;
the lamp carried by the lamp housing and adapted to emit a light having a wavelength between 10 nanometers and 380 nanometers; and
a control unit adapted to be positioned entirely in an exterior of the air duct and adapted to provide power to the lamp, comprising:
a ballast adapted to regulate the power provided to the lamp, and
a control housing adapted to carry the ballast and comprising a plurality of mounting apertures, wherein each of the plurality of mounting apertures is adapted to receive a fastener to secure the control housing to the lamp housing.
